(19) United States
(12) Patent Application Publication
(10) Pub. No.: US 2010/0021598 A1
(43) Pub. Date: Jan. 28, 2010

(54) OZONATED LIQUID DISPENSING UNIT

(76) Inventor: Daniel W. Lynn, Omaha, NE (US)

Correspondence Address: POLSINELLI SHUGHART PC, 700 W. 47TH STREET, SUITE 1000, KANSAS CITY, MO, 64112-1802, US

(21) Appl. No.: 12/179,335

(22) Filed: Jul. 24, 2008

(51) Int. Cl. C11D 3/04; B01J 19/00; B01J 19/08; A61L 2/18; A23L 3/3454

(52) U.S. Cl. 426/323; 422/189; 422/186.12; 422/29; 510/218

(57) ABSTRACT

A ozonated liquid dispensing unit is described. The unit produces and dispenses an ozonated liquid that may be used to clean and sanitize a variety of articles or used in conjunction with cleaning processes and other apparatus. The unit includes a liquid input port to receive liquid into the unit. The unit includes a first dielectric cell for producing ozone gas from ambient air and a second dielectric cell for producing ozone gas. The first dielectric cell is in supply communication with the second dielectric cell for supplying the second dielectric cell with a supply gas containing the ozone gas generated from the ambient air. The second dielectric cell produces ozone gas from the supply gas. An injector is in fluidic communication with the liquid input port. The injector in supply communication with the second dielectric cell for receiving the ozone gas from the second dielectric cell, and the injector mixes the ozone gas from the second dielectric cell with the liquid from the liquid input port to produce an ozonated liquid. A liquid output port discharges the ozonated liquid from the unit. A faucet or spray may be used to control the discharge of the ozonated liquid from the unit.

### FIELD OF INVENTION

The present invention relates to an ozonated liquid dispensing unit that produces and dispenses an ozonated liquid that may be used to clean and sanitize a variety of articles or used in conjunction with cleaning processes and other apparatus.

### BACKGROUND OF INVENTION

Prior attempts to provide an ozonated liquid in a kitchen environment have failed to provide an ozonated liquid with sufficient concentrations of ozone resulting in poor cleaning and sanitizing. Without sufficient ozone concentration, conventional cleaning and sanitizing methods may still be necessary at extra labor, equipment, and supply costs.

Other prior attempts to provide an ozonated liquid have involved electrochemical ozone generation. Such systems are difficult to maintain.

Other prior attempts to provide an ozonated liquid have involved systems too large and too bulky to be effectively used in some residential or commercial applications. Many of these systems are also too expensive for use in the home or are not economical to be used in commercial applications.

Other prior attempts to provide an ozonated liquid have involved systems requiring significant mechanical alterations to existing water supply and delivery systems. Some systems also require the output of ozone gas to be adjusted each time the system is turned on. Further, many previous systems cannot be used with multiple, different dispensing applications.

Other prior attempts to provide an ozonated liquid have involved systems that create too much off-gassing of ozone. Although ozone gas is generally harmless, OSHA workplace requirements require that ozone levels are maintained below certain minimums.

### SUMMARY OF INVENTION

An ozonated liquid dispensing unit is described herein. The unit produces and dispenses an ozonated liquid that may be used to clean and sanitize a variety of articles or used in conjunction with cleaning processes and other apparatus. The unit is compact, may be conveniently installed in a commercial or residential kitchen, restroom or other area with a water supply. The units provides an ozonated liquid with a high concentration of ozone gas sufficient to clean and sanitize food items, food preparation items, food preparation surface, bathrooms, medical equipment, drains and to provide for hand-washing and hygiene needs. The unit uses multiple dielectric cells in an in-line configuration to create the ozone gas that is mixed with the water to form the ozonated liquid.

Foods, food preparation areas as well as other surfaces that may benefit from sterilization provided by the unit. In the food industry, the ozonated liquid from the unit provides for chemical-free sterilization of contaminated surfaces and tools, such as those used in the processing of raw meat. The ozonated liquid cleans toxic substances 3,000 times faster than chlorine, and unlike chlorine, ozonated liquid is completely safe and natural. The ozonated liquid kills micro-organisms, including E. coli, salmonella, bacteria, viruses, molds, etc. The ozonated liquid also remove pesticides and other residues from fruits and vegetables. The ozonated liquid also reduces odors in the environment on which the ozonated liquid is sprayed. The unit is ideal for residential food preparation, commercial food preparation, or any place a sterile, cleaning solution is needed. In a commercial setting, fruits and vegetables may be washed with the unit and its ozonated liquid to increase the shelf-life of the items. By removing the micro-organisms from the surfaces of the fruit and vegetables that may cause decay and spoilage, the fruit and vegetables will not decay or spoil as fast.

The ozonated liquid dispensing unit includes a liquid input port to receive the liquid, such as water, into the unit to be mixed with ozone gas to form the ozonated liquid. The unit includes a first dielectric cell for producing ozone gas from ambient air and a second dielectric cell for producing ozone gas. The first dielectric cell is in supply communication with the second dielectric cell for supplying the second dielectric cell with a supply gas comprising the ozone gas generated from the ambient air. The second dielectric cell produces ozone gas from the supply gas. An injector is in fluidic communication with the liquid input port. The injector in supply communication with the second dielectric cell for receiving the ozone gas from the second dielectric cell, and the injector mixes the ozone gas from the second dielectric cell with the liquid from the liquid input port to produce an ozonated liquid. A liquid output port discharges the ozonated liquid from the unit. A faucet or spray may be used to control the discharge of the ozonated liquid from the unit.

The unit is easy to install. Generally, the unit is just plugged into an electrical unit and a water supply is provided to the unit. The unit discharges the ozonated liquid into a liquid supply line in fluidic communication with a sprayer or faucet. A handle, knob or other actuator is manipulated in order to begin the production and flow of ozonated liquid from the unit.

Ozone gas is unstable, which provides for it cleaning and sanitizing capabilities, but also makes consistent ozone levels difficult to maintain when the gas is mixed into a solution. Ozone gas cannot be packaged or stored and must be generated on site. The unit reduces the need for chemicals, hot water, and labor. Conventional cleaning systems often require the use of warm or hot water, which may form condensation in the surrounding workspace. This condensation may provide for or encourage the growth of microorganisms. Because unit only uses cold water, condensation is less likely to form in the surrounding workspace. The unit also reduces the hydraulic load on the waste-water treatment system and eliminates the need to treat the chemicals that would be present in conventional wastewater discharge streams.

Ozone creates none of the trihalomethanes commonly associated with chlorine compounds. When properly matched to the application, ozone will reduce most organic compounds to carbon dioxide, water and a little heat. Finally, as ozone sheds the atom of the oxygen causing its molecular instability during the oxidation process, it becomes oxygen again.

### DESCRIPTION OF FIGURES

FIG. 1 shows a perspective view of the ozonated liquid dispensing unit with the output side of the unit visible.

FIG. 2 is a perspective view of the ozonated liquid dispensing unit with the input side of the unit visible.

FIG. 3 is a plan diagram showing the installation of the ozonated liquid dispensing unit in conjunction with a sink and faucet.

FIG. 4 is a front view of the ozonated liquid dispensing unit with the housing cover removed.

FIG. 5 is a top-down view of the ozonated liquid dispensing unit with the housing cover removed.

FIG. 6 is a rear view of the ozonated liquid dispensing unit with the housing cover removed.

FIG. 7 is a view showing a sprayer attached to the ozonated liquid dispensing unit.

FIG. 8 is a view of the first and second dielectric cells.

### DETAILED DESCRIPTION OF INVENTION

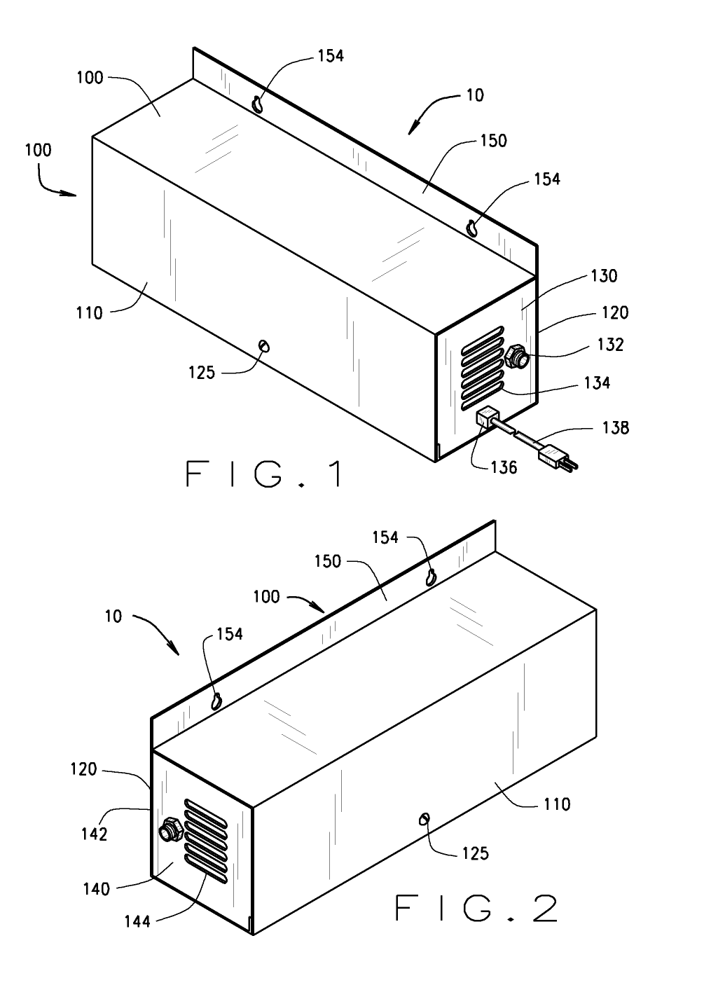

An ozonated liquid dispensing unit is described herein. With reference to FIGS. 1 and 2, an ozonated liquid dispensing unit 10 is shown. The unit 10 includes a housing 100, a removable housing cover 110 and a housing support 120. The housing 100, the housing cover 110, and the housing support 120 form a rectangular, box-like structure that houses the internal components of the unit 10. The housing 100 may be designed or engineered in other shapes and configurations. The housing 100, the housing cover 110, and the housing support 120 are made from sturdy or rugged materials, such as stainless steel, aluminum, or metals. Plastics and other composite materials may also be utilized in the construction of the housing 100, the housing cover 110 and the housing support 120.

As shown in FIGS. 4-6, the housing cover 110 is removed from the housing 100 to show the housing support 120, which receives and stabilizes the internal components of the unit 10. The housing cover 110 may be secured to the housing support 120 via securing means 125, such as a screw, pin, latch, lock, or other connection means for suitably attaching the housing cover 110 to the housing support 120 in a removable fashion.

FIG. 1 shows an output side 130 of the unit 10. The output side 130 includes a liquid output port 132 and an output side vent 134, and an electrical supply connection 136. The liquid output port 132 dispenses the ozonated liquid prepared in the unit 10 from the unit 10. The output side vent 134 assists in dissipating heat produced in the housing 100 from the electrical generation of ozone gas. The electrical supply connection 136 is in electrical communication with an electrical supply 138 to provide power to the unit 10.

FIG. 2 shows an input side 140 of the unit 10. The input side 140 is generally opposite of the output side 130. The input side 140 includes a liquid input port 142 and an input side vent 144. The liquid input port 142 includes threadable connections to receive a liquid input line 200 that supplies the unit 10 with water that is to be mixed with the ozone gas. The liquid input line 200 is threadably received by the liquid input port 142.

Ozonated liquid prepared by the unit 10 is discharged by the unit 10 from the liquid output port 132. A liquid output line 210 is connected to the liquid output port 132. The liquid output port 132 may include threadable connections for connecting the liquid output line 210 to the liquid output port 132. The liquid output line 210 supplies, for example, an ozone faucet 233 or other sprayer means, with a supply of the ozonated liquid.

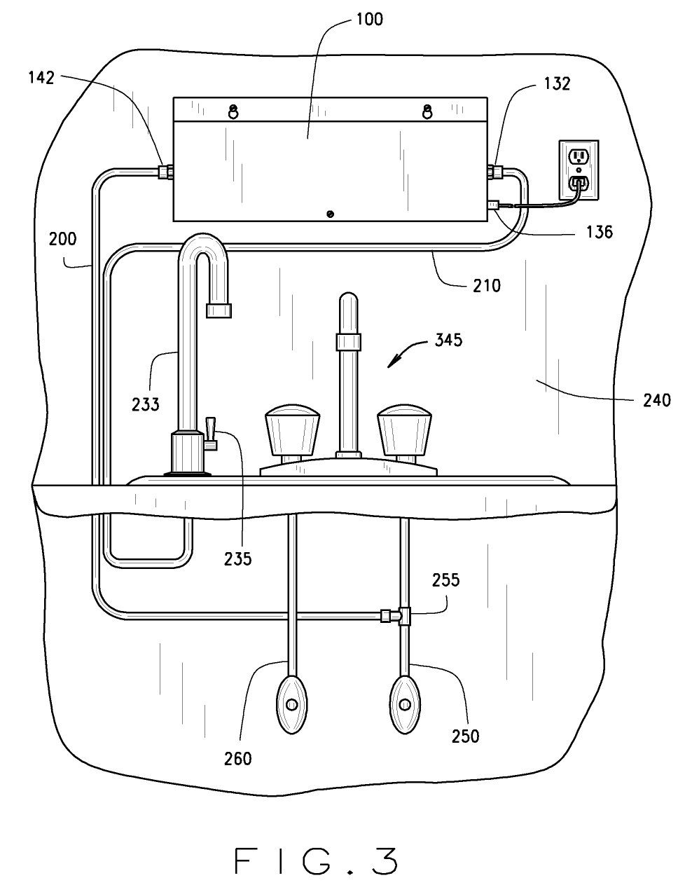

The unit 10 may be conveniently mounted adjacent to or over a faucet/sink combination 345, such as shown in FIG. 3. The ozonated liquid may be disposed through the drain into existing waste water systems and the municipal sewer systems.

In the embodiments shown, the housing support 120 forms a flange 150 that extends beyond the housing cover 110. The flange 150 includes openings 154 which may be used to affix the unit 10 to a wall, cabinet or other structure via bolts, screws, rivets or other fastening means.

In other embodiments, the unit 10 may be placed onto a counter or underneath a counter in, for example, a kitchen cabinet or other storage area. FIG. 3 shows a diagram of unit 10 installed at the faucet/sink combination 345. During a typical installation of the unit 10, a t-shaped adaptor 255 is placed in the cold water supply 250. The adaptor 255 branches the cold water from the cold water supply 250 to the unit 10, while still providing normal cold water to the faucet/sink combination 345. The adapter 255 supplies fresh, cool water via the liquid input line 200 to the unit 10. The adapter 255 does not interrupt flow of the cool water supply 250 to the faucet/sink combination 345. A hot water supply 260 typically does not receive, or is involved with, the preparation of the ozonated liquid by the unit 10.

As ozone gas is created by the unit 10 and the ozone gas is mixed into the cold water entering the unit 10 from the cold water supply 250, the ozonated liquid is discharged at the liquid output port 132. The liquid output port 132 is in fluidic communication with an ozone faucet 233 via the liquid output line 210. By turning on a handle 235 of the ozone faucet 233, water is drawn into and through the unit 10 where ozonated gas prepared in the unit 10 is mixed with the water. During operation of the unit 10, the operator only needs to pull on the handle 235 in order for ozonated liquid to be discharged from the ozone faucet 233. The unit 10 does not require other manual actuation each time the unit 10 is used, i.e., the operator need not actuate an on/off switch or the like.

The internal components of the unit 10 are shown in FIGS. 4-6. Water from the cool water supply 250 enters a fluid flow switch 215, which activates the unit 10 upon sensing a flow of water. The fluid flow switch is in fluidic communication with an injector 252 via a liquid line 218.

The liquid line 218 fluidly connects the fluid flow switch 215 with the injector 252. The liquid line 218 may comprise a hose, plastic tubing, metal braided tubing, or other suitable structure for communicating liquid from the fluid flow switch 215 to the injector 252.

The water supplied to the injector 252 is mixed with ozone gas from the first dielectric cell 220 and the second dielectric cell 240. As further described herein, the first dielectric cell 220 supplies supply gas containing ozone gas to the second dielectric cell 240. The second dielectric cell 240 creates additional ozone gas in the supply gas and supplies the ozone gas to the injector 252, and the injector 252 mixes the ozone gas into the water in order to form the ozonated liquid that is discharged from the unit 10 at the liquid output port 132.

The injector 252 forms the ozonated liquid by mixing ozonated gas with the water. Suitable injectors are commercially available from the Mazzei Injector Corporation. The injector 252 uses a pressure differential between the water entering the injector 252 from the liquid line 218 and the fluid exiting the injector 252 to mix the water with ozone gas. The pressure at an inlet port of the injector 252 is higher than the pressure at an outlet port of the injector 252, and this pressure differential creates a suction in the injector 252 that draws the ozone gas from the second dielectric cell 240 into the injector 252 for mixing with the water.

An important feature of the unit 10 is the use of multiple dielectric cells, namely, the first dielectric cell 220 and the second dielectric cell 240. The first dielectric cell 220 prepares ozone gas that is supplied to the second dielectric cell 240, which creates additional ozone gas, thus creating a highly concentrated supply of ozone gas that is supplied to the injector 250. In other embodiments, additional dielectric cells may be employed.

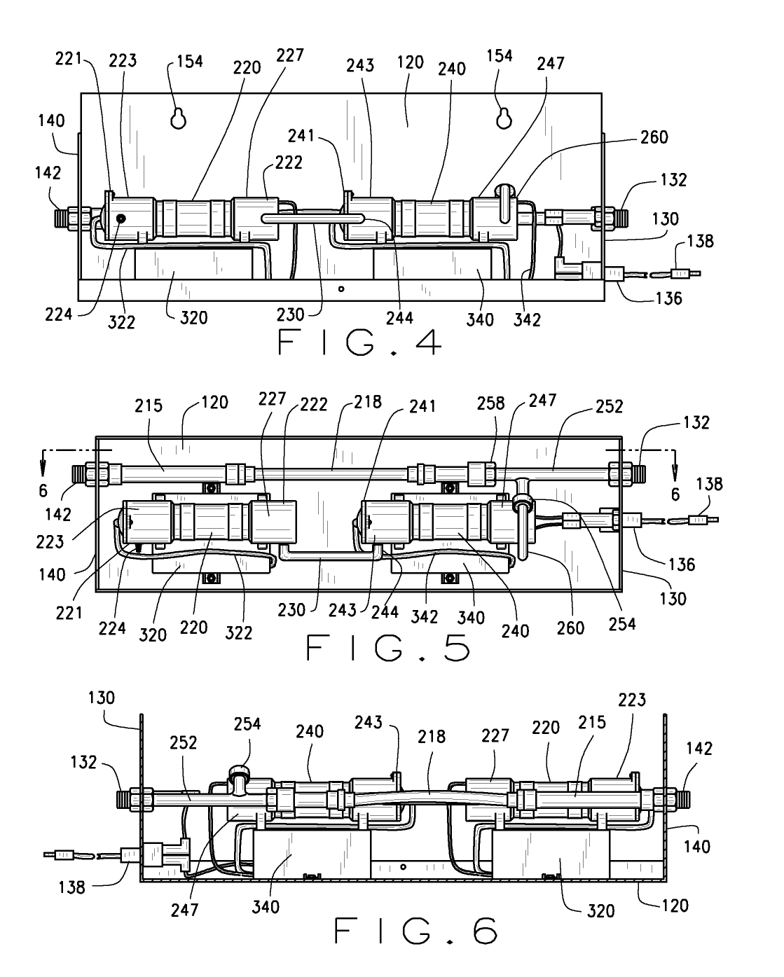

With reference to FIG. 5, top-down view of the unit 10 is shown. Ambient air is drawn into the first dielectric cell 220 via an ambient air input 224 of a first gas input trap 223. The first gas input trap 223 is sealingly connected to and surrounds a first end 221 of the first dielectric cell 220. The first dielectric cell 220 makes ozone gas from the ambient air passing through the first dielectric cell 220.

The first dielectric cell 220 includes a glass or other insulating cylinder. An electrical conductor passes through the cylinder. A conductive metal lattice, metal mesh, or coil wire surrounds the conductor. When power is supplied to the first dielectric cell 220, electricity passes through the conductor and sparks and arcs. This electrical discharge splits the oxygen molecules creating ozone gas from the oxygen molecules present in the ambient air inside of the dielectric cell 220. This method is generally referred to as corona discharge. The second dielectric cell 240 is constructed similar to the first dielectric cell 220.

As described above, ozone gas created by the coronal discharge in the first dielectric cell 220 is captured and supplied to the second dielectric cell 240. The supply gas from the first dielectric cell 220 to the second dielectric cell 240 contains an amount of ozone gas. A second or output end 222 of the first dielectric cell 220 is sealingly connected to and surrounded by a first gas output trap 227. The first gas output trap 227 funnels the ozone gas created by the first dielectric cell 220 to a first gas line 230 which is in fluidic communication with a second gas input trap 243 and an ozone gas input 244 on the second gas input trap 243. The first gas line 230 thus connects to the first gas output trap 227 to the ozone gas input 244. The second gas input trap 243 is sealingly connected to a first or input end 241 of the second dielectric cell 240. As such, supply gas to the second dielectric cell 240 already includes a first amount of ozone gas. The supply gas from the first dielectric cell 220 is further processed by the second dielectric cell 240 to add an additional amount of ozone gas to the supply gas.

The first gas output trap 227 seals the output of ozone gas from the first dielectric cell 220 such that nearly all of the ozone gas created by the first dielectric cell 220 or the output of gas from the first dielectric cell 220 is supplied in a closed communication via the first gas line 320 to the second dielectric cell 240. The closed communication provides for the second dielectric cell 240 to form ozone gas from the output gas of the first dielectric cell 220.

The ozonated gas produced by the second dielectric cell 240 is transported via a second gas line 260 to an injector gas input port 254 of the injector 252. The second gas output trap 247 is sealingly connected to a second or output end 242 of the second dielectric cell 240.

The use of the first and second dielectric cell 220 and 240 creates an increased concentration of ozone gas in supply communication with the injector 252. A single dielectric cell similar to the first dielectric cell 220 or the second dielectric cell 240 creates ozone gas at a concentration of 0.5 parts per million. However, the use of two of the two inline dielectric cells, i.e., the first dielectric cell 220 and the second dielectric cell 240, creates a supply of ozone gas to the injector 252 having a concentration of approximately 1.3 ppm.

The unit 10 is electrically connected to the power supply 138, such as a 115-volt power supply. The electrical connector 136 of the unit 10 is in electrical communication with a first power supply 320 and a second power supply 340. A first electrical supply line 322 is in electrical communication with the first power supply 320 and at a conductor positioned at the first end 221 of the first dielectric cell 220. A second electrical supply line 342 is in electrical communication with the second power supply 340 and at a conductor positioned at the first end 241 of the second dielectric cell 240. The electrical supply lines 322 and 342 provide the electricity for the corona discharge.

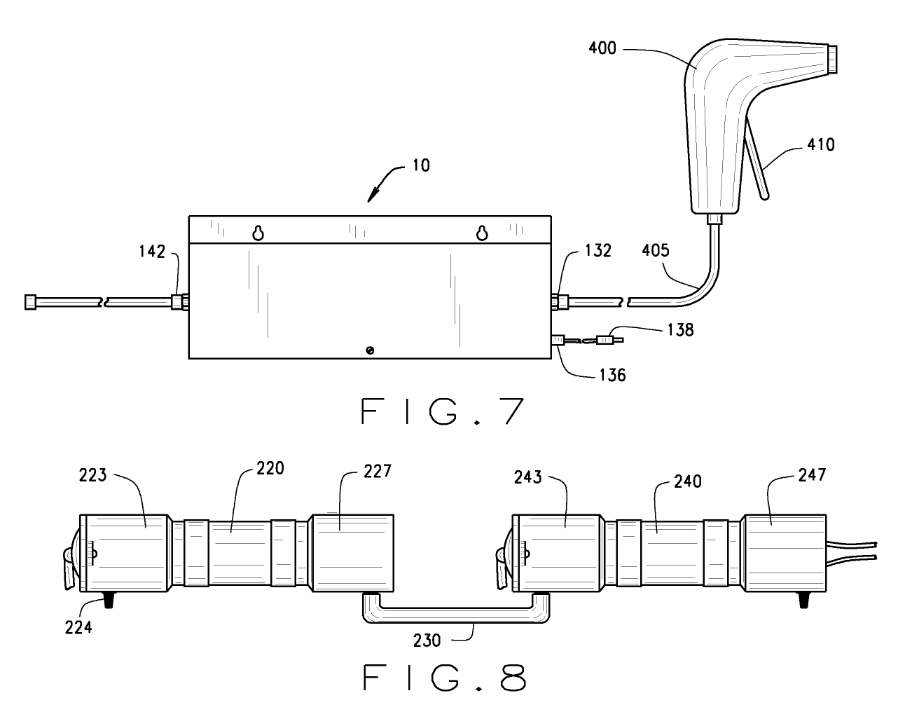

Turning now to FIG. 7, the ozone faucet 233 has been replaced with a spray nozzle 400 having a handle 410 to actuate the discharge of the ozonated liquid. The spray nozzle 400 is in fluidic communication with the liquid output port 132. A hose, tube or other liquid communication structure 405 is used to supply the sprayer 400 with the ozonated liquid from the liquid output port 132. The spray nozzle 400 or the liquid communication structure 405 includes a valve means or other shut-off to control the output of liquid from the spray nozzle. For example, a handle 410 of the spray nozzle 400 may actuate the valve or otherwise control the flow of the ozonated liquid from the spray nozzle 400. The spray nozzle 400 may be used to spray fruits and vegetables in order to kill microorganisms, remove dirt and debris, and/or wash of pesticide residue.

The spray nozzle 400 may further be used to clean and sanitize shower areas and rest rooms. Spraying the ozonated liquid onto such bathroom surfaces is an economical and convenient method to provide for sanitation. The ozonated liquid does not leave a residue or film on the restroom and shower surfaces. No other chemicals or detergents are required. There is no clean-up or storage of soiled conventional cleaning tools, such as a mop or mop bucket.

The unit 10 provides a flow of ozonated liquid at approximately 25 psi and 1.5 gallons per minute from the ozone faucet 233 or the spray nozzle 400. The ozonated liquid has an ozone concentration of approximately 1.8 parts per million.

The unit 10 also finds utility in cleaning fruits and vegetables. Herbicide residue may be removed from the fruit and vegetable surfaces. Pathogens, such as salmonella, may be easily removed from more delicate food surfaces, such as that of a tomato. Raw meats and carcasses and may also be directly contacted with the ozonated liquid.

The unit 10 may also be used to clean and sterilize medical instruments. The unit 10 may also be used for general hand-washing and wound-flushing. The unit 10 may also be used for drain cleaning. The oxidation provided by the ozonated liquids will break-up many deposits in drains.

In operation of the unit 10, the user actuates the handle 235 of the ozone faucet 233. When the cold water begins to flow through the liquid input line 200 to the unit 10, the liquid flow switch 215 activates the first power supply 320 and the second power supply 340 to discharge electrical current to the first dielectric cell 220 and the second dielectric cell 240 to the begin creation of ozone gas. Generally, the operator should expect to wait several seconds for the water flowing from the ozone faucet 233 to transition to ozonated liquid. When the handle 235 is turned off, water flow through the unit 10 is stopped and the liquid flow switch 215 turns the first power supply 320 and the second power supply 340 off.

Those skilled in the art will appreciate that variations from the specific embodiments disclosed above are contemplated by the invention. The invention should not be restricted to the above embodiments, but should be measured by the following claims.

What is claimed:

1. An ozonated liquid dispensing unit, comprising:
a liquid input port to receive liquid into the unit;
a first dielectric cell for producing ozone gas from ambient air;
a second dielectric cell for producing ozone gas;
the first dielectric cell in supply communication with the second dielectric cell for supplying the second dielectric cell with a supply gas comprising the ozone gas generated from the ambient air, and the second dielectric cell produces ozone gas from the supply gas;
an injector in fluidic communication with the liquid input port;
the injector in supply communication with the second dielectric cell for receiving the ozone gas from the second dielectric cell, wherein the injector mixes the ozone gas from the second dielectric cell with the liquid from the liquid input port to produce an ozonated liquid; and
a liquid output port to discharge the ozonated liquid from the unit.

2. The unit according to claim 1, wherein the first dielectric cell includes a first gas output trap sealingly connected to the first dielectric cell.

3. The unit according to claim 2, wherein the first gas output trap is in supply communication with the second dielectric cell.

4. The unit according to claim 3, wherein the second dielectric cell receives a supply gas containing ozone gas from the first dielectric cell and creates additional ozone gas from the supply gas.

5. The unit according to claim 1, wherein the first dielectric cell and second dielectric cell are in a closed communication to supply ozone gas from the first dielectric cell to the second dielectric cell.

6. The unit according to claim 1, wherein the injector includes a liquid input port in fluidic communication with a fluid flow switch, wherein the injector also includes a gas input port in communication with the second dielectric cell.

7. The unit, according to claim 1, wherein the power supply includes a first power supply that supplies power to the first dielectric cell and a second power supply that supplies power to the second dielectric cell.

8. The unit according to claim 1, wherein the housing comprises a housing cover and a housing support.

9. The unit according to claim 8, wherein the first dielectric cell and the second dielectric cell are mounted to the housing support.

10. The unit according to claim 1, wherein the housing includes a liquid input port in fluidic communication with a supply of cool water and an ozonated liquid output port in fluidic communication with a faucet, sprayer, or other liquid dispensing device.

11. The unit according to claim 1, wherein the first dielectric cell draws a supply gas of the ambient air through a first gas input trap having an ambient air input; the first dielectric cell creates ozone gas from the ambient air drawn in through the ambient air input; a first gas output trap is sealingly connected to the first dielectric cell, the first gas output trap in communication with a second gas input trap sealingly connected to the second dielectric cell; a second gas output trap sealingly connected to the second dielectric cell, the second gas output trap in communication with a gas line, the gas line in communication with the injector to supply the injector with ozone gas.

12. The unit according to claim 1, wherein a flow of water activates a liquid flow switch that activates a first power supply and a second power supply.

13. The unit according to claim 1, wherein the ozonated liquid has an ozone concentration of approximately 1.8 parts per million.

14. The unit according to claim 1, further comprising a faucet or sprayer in fluidic communication with the liquid output port, and actuation of the faucet or sprayer result in liquid flow in the unit that activates the first dielectric cell and the second dielectric cell.

15. The unit according to claim 1, wherein the first dielectric cell and the second dielectric cell comprises an electrical conductor surrounded by glass to create ozone gas through corona discharge.

16. An ozonated liquid dispensing unit, comprising:
a liquid input port to receive water into the unit from a cool water supply;
a first dielectric cell for producing ozone gas from ambient air, the first dielectric cell comprising an output trap sealingly connected to an output end of the first dielectric cell;
a second dielectric cell for producing ozone gas;
the output trap of the first dielectric cell in closed communication with an input trap of the second dielectric cell for supplying the second dielectric cell with a supply gas comprising ozone gas generated from the ambient air at the first dielectric cell, and the second dielectric cell produces additional ozone gas from the supply gas;
an injector in fluidic communication with the liquid input port;
the injector in supply communication with the second dielectric cell for receiving the ozone gas from the first and second dielectric cells, wherein the injector mixes the ozone gas from the first and second dielectric cells with the water from the liquid input port to produce an ozonated liquid; and
a liquid output port to discharge the ozonated liquid from the unit.

17. A method of forming and dispensing an ozonated liquid, comprising
providing a first dielectric cell for producing ozone gas from ambient air;
providing a second dielectric cell for producing ozone gas, the first dielectric cell in supply communication with the second dielectric cell;
creating a supply gas containing ozone gas in the first dielectric cell from ambient air;
supplying the second dielectric cell with the supply gas;
creating additional ozone gas in the second dielectric cell from the supply gas;
directing water to an injector, the injector in supply communication with the second dielectric cell for receiving the ozone gas from the second dielectric cell;
mixing the ozone gas from the second dielectric cell with the water to produce an ozonated liquid; and
dispensing the ozonated liquid from the unit.

18. The method according to claim 17, further comprising providing a first gas output trap sealingly connected to the first dielectric cell, and the first gas output trap in communication with an input trap of the second dielectric cell

19. The method according to claim 17, further comprising detecting a flow of water and activating the first dielectric cell and the second dielectric cell after detecting the flow of water.

20. The method according to claim 17, further comprising spraying a food item, a food preparation item, a food preparation surface, a bathroom, or a drain with the ozonated liquid.
